(19) United States
(12) Patent Application Publication
(10) Pub. No.: US 2002/0161289 A1
(43) Pub. Date: Oct. 31, 2002

(54) Detector array for optical spectrographs

(76) Inventors: George W. Hopkins II, Sunnyvale, CA (US); Ganapati R. Mauze, Sunnyvale, CA (US); Tirumala R. Ranganath, Palo Alto, CA (US)

Correspondence Address: Agilent Technologies, Inc., 1601 California Ave., MS 17L-5A, Palo Alto, CA, 94304-1111, US

(21) Appl. No.: 09/846,489

(22) Filed: Apr. 30, 2001

(51) Int. Cl. G01N 33/48

(52) U.S. Cl. 600/322; 356/39

(57) ABSTRACT

A composite detector device that incorporates a plurality of types of detector elements to cover a broad wavelength range. There may be one or more individual detector elements of each detector type. The individual detector elements are positioned upon a single substrate so that when light from a sample passes through a spectral dispersing element, each detector element is exposed to light of a predetermined, limited wavelength range.

### DESCRIPTION

1. Field of the Invention

The invention relates generally to optical detection instrumentation design. The invention more specifically relates to optimized configurations of individual optical sensors in monolithic packages in instruments for non-invasive optical sensing of samples, e.g. blood glucose concentration.

2. Background of the Invention

The first photoelectric spectrographs used a single photodetector. Typically, the spectrum was scanned across this single photodetector, allowing sequential detection of light at different wavelengths. W. W. Coblentz first used this technique around 1905, and it is still in use today. Multiple discrete detectors can be used to give a multichannel advantage in detecting a spectrum. U.S. Pat. No. 5,011,284 to Tedesco uses an array of a plurality of photosensitive detectors in a Raman scattering device. Multiple discrete detectors are readily available, in single materials, in monolithic form.

The first scanned array detectors were commercially available in the early 1970s. This type of detector, called a photo diode array (PDA), is now widely used in spectrographs. It has the advantage of detecting a large number of wavelengths simultaneously, but these detectors are typically scanned at a constant rate. The first such detectors used silicon technology to cover the spectral range from 200 nm to 1100 nm. Such detectors are now available in indium gallium arsenide (InGaAs) form from, e.g. Sensors Unlimited, Inc. (Princeton, N.J.) to cover 900 nm to 2500 nm by using different alloys to cover different segments of this spectrum. These detectors are available only in the PDA form, which has switching noise. Silicon detectors are also available in charge-coupled device (CCD) form, which has no switching noise. All of these detector arrays are on a single substrate and can be cooled by a thermoelectric (TE) cooling device.

Various methods of combining components or circuits on like or different materials are known in the art. U.S. Pat. No. 5,670,817 to Robinson describes a combination of radiation detectors and readout circuits in monolithic array form on a single semiconductor substrate material. When circuits or components are combined on different materials in a device, the differing thermal coefficients of expansion of the materials over the operating temperature range of the device lead to stresses on the materials. U.S. Pat. No. 5,672,545 to Trautt et al. teaches the use of compensation layers on one of the substrate materials to reduce the effect of temperature-related stresses. U.S. Pat. No. 5,565,675 to Phillips describes a mechanically stable mount for a discrete optical receiver above a wiring board having various readout componentry.

### Non-Invasive Spectrometric Blood Chemistry Sensing

Tests to measure blood chemistry frequently involve obtaining a sample through an invasive procedure. Development of non-invasive testing method has become an important topic in recent years due to the perceived potential for the spread of life threatening diseases such as acquired immunodeficiency disease syndrome (AIDS), hepatitis, and other similar blood diseases. For example, a recent article, “Nosocomial transmission of Hepatitis B virus associated with the use of a spring-loaded finger-stick device,” New England Journal of Medicine 326 (11), 721-725 (1992), disclosed a mini-hepatitis epidemic in a hospital caused by the improper use of an instrument for taking blood samples. Hospital personnel were unintentionally transmitting hepatitis from one patient to another via the sampling device. This type of disease transfer is eliminated with non-invasive testing.

The diabetic population has also been clamoring for non-invasive test instruments. Many diabetics must test their blood glucose levels four or more times a day. The modern battery powered instruments for home use require a finger prick to obtain the sample. The extracted blood sample is then placed on a chemically treated carrier which is inserted into the instrument to obtain a glucose reading. This finger prick is painful and can be a problem when required often. In addition, the cost for the disposable test materials and the mess and health risks associated with having open bleeding is undesirable.

Accordingly, much work has been done on non-invasive blood analyte sensing, that is, without penetration of the skin and without withdrawing a blood sample from the body. WIPO application WO 97/30629 describes a method and apparatus for non-invasive blood glucose sensing. U.S. Pat. No. 5,086,229 to Rosenthal et al. describes using near infrared (NIR) light to obtain information about blood glucose concentration non-invasively. U.S. Pat. No. 5,460,177 to Purdy et al. teaches a method for non-invasive measurement of concentration of analytes in blood using continuous radiation spectrum and measuring a plurality of wavelength ranges. U.S. Pat. No. 5,424,545 to Block et al. describes another method of non-invasive blood analyte sensing by making non-spectrometric infrared measurements of the radiation emanating from a sample illuminated with multiple incident beams of radiation. U.S. Pat. No. 5,710,630 to Essenpreis et al. discloses an interferometric method said to be useful in non-invasive determination of glucose concentration. U.S. Pat. No. 5,692,504 to Essenpreis et al. measures light transit time within a biological matrix as an indicator of glucose concentration within the matrix. Optical rotation of light is used in U.S. Pat. No. 5,209,231 for non-invasive measurement of glucose concentration. U.S. Pat. No. 5,703,364 to Rosenthal describes a method of improving accuracy of near infrared (NIR) quantitative measurements by illuminating a sample with multiple NIR light sources and controlling the length of time each source is on.

Several companies have been developing non-invasive spectrometers for obtaining blood chemistry information. Nonin Medical, Inc. (Plymouth, Minn.) produces devices to measure blood gas composition which have a plurality of light sources and detectors. See, e.g. U.S. Pat. 4,773,422. Biocontrol Technology, Inc. (Pittsburgh, Pa.) and Futrex, Inc. (Gaithersburg, Md.) are each reported to be investigating the use of near-IR spectral sensors to measure glucose levels. The near infrared spectral region (700-1100 nm) contains the third overtones for the glucose spectrum and eliminates many of the water bands and other inference bands that are potential problems for detection. This work has been carried out using classic spectrophotometric methods such as scanning spectrophotometers which scan wavelength by wavelength across a broad spectrum. The data obtained from these methods are spectra which then require substantial data processing to eliminate background; accordingly, the papers are replete with data analysis techniques utilized to glean the pertinent information. Examples of this type of testing includes the work by Clarke, see U.S. Pat. No. 5,054,487; and by Rosenthal et al., see e.g., U.S. Pat. No. 5,028,787. Although the Clarke work uses reflectance spectra and the Rosenthal work uses primarily transmission spectra, both rely on obtaining near infrared spectrophotometric data.

The extraction of information about blood glucose concentration from spectral or other data received by the detector is a complex problem due to the presence of components other than glucose in the area that is being sensed. These other components give rise to their own signals, which may be much larger in magnitude than the signal from glucose. Various methods have been described for obtaining relevant information in the presence of competing signals. U.S. Pat. No. 5,321,265 to Block discloses a non-invasive testing method using multiple detecting units, each covering a broad and overlapping region of the detected spectrum. The Block patent analogizes the process to the ability by humans to perceive subtle differences in color using only three different color receptors in the eye. Rosenthal et al. disclose their own method for analyzing spectral data in U.S. Pat. No. 5,028,787. U.S. Pat. No. 5,070,874 to Barnes et al. discloses a method for non-invasive determination of glucose concentration in a patient's body by manipulating spectral data observed over a single wavelength range. U.S. Pat. No. 5,242,602 describes the use of chemometric methods and algorithms for the analysis of multi-analyte systems.

Generally, in photometric analysis of complex samples, it is useful to collect data over a broad range of wavelengths. However, the useful range of a detector is limited. A single type of detector or detector alloy is unable to cover the broad range of wavelengths extending from the ultraviolet region through the visible region and into the IR region of the spectrum. However, because of the different substrate materials needed to provide detection over such a broad wavelength range, the manufacture of such a device is difficult. Moreover, because of the need to scan every sensing element in order to read an array, there is a tradeoff between the time it takes to sample any given sensing element and the total amount of time it takes to scan the array. This may prevent using a fast scan rate. Processing a full spectrum of data with each full scan may further reduce performance of the instrument.

### SUMMARY OF THE INVENTION

A new type of detector device has now been designed which meets the above requirements. The detector device is single composite unit that incorporates a plurality of types of detector elements to cover a broad wavelength range. There may be one or more individual detector elements of each detector type. The individual detector elements are positioned upon a single substrate so that when light from a sample passes through a spectral dispersing element, each detector element is exposed to light of a predetermined wavelength range.

Also herein disclosed is a method and apparatus for non-invasive determination of one or more blood chemistry measurements from a subject using the detector device described herein. The method includes shining light upon a subject's body, providing an optical system to confer light received from the patients body onto the detector device of the current invention, receiving information about a limited number of wavelength regions of light from the detector device, and determining the blood chemistry measurement from the information received from the detector device. The wavelength regions are predetermined for increasing or optimizing the information obtained about the blood chemistry of the subject while reducing or minimizing the number of optical sensing elements in the optical detector device. In one embodiment the apparatus for non-invasive determination of one or more blood chemistry measurements is small enough to be easily wielded in one hand and to be easily portable.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 is a highly schematic diagram of a composite detector array.

FIG. 2 schematically depicts an instrument for non-invasive measurement of blood chemistry using a composite detector array according to the invention.

### DETAILED DESCRIPTION

Further aspects and advantages of the invention will be set forth in part in the description which follows, and in part will be obvious from the description, or may become readily apparent through practice of the invention. It is to be understood that both the foregoing general description and the following detailed description are exemplary and explanatory only and are not restrictive of the invention, as claimed. It must be noted that, as used in the specification and the appended claims, the singular forms “a”, “an” and “the” include plural referents unless the context clearly dictates otherwise. Thus, for example, reference to “a material” includes mixtures of materials, reference to “a chamber” includes multiple chambers, and the like.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

“Invasive procedures,”″ as used herein are procedures where a sample such as blood is taken from the body by puncture or other entry into the body before analysis, while noninvasive procedures do not require bodily penetration.

“Light” is used herein in a broader sense than just electromagnetic energy visible to the human eye—it includes spectral energy through the UV, visible, and infrared range of the spectrum, generally from wavelengths of about 100 nanometers to about 40 micrometers.

“Detector type” refers to the material that forms the active portion of the detector element, or which forms the photosensitive portion of the detector element. “Detector element” refers to the smallest section of the detector device that can be interrogated by the surrounding circuitry to return a signal related to the light incident upon the detector element. Detector elements are chosen according to their response characteristics for the wavelength range of light to be detected. Response characteristics for detector elements are generally published or readily ascertainable, thus selecting appropriate detector types for detecting particular wavelength ranges is well within the ordinary skill of those knowledgeable in the art. “Composite” refers to the manufacture of a device using multiple dissimilar photosensitive materials which function to measure light. The dissimilar photosensitive materials may be sensitive to light in differing portions of the spectrum, thus allowing the composite detector to measure light over a broader spectral range than possible by any single photosensitive material.

The invention provides a single-package solution to the problem of broad spectrum, multiple discrete sensing of light: In this context, “broad spectrum” means that the detector elements may be of different types which allow sensing of light over a range of from about 100 nm to about 40 micrometers, or, more particularly, from about 200 nm to about 26 micrometers, or yet more particularly, from about 300 nm to about 11 micrometers. “Multiple” in this context means that a plurality of limited wavelength regions of light are detected. “Discrete” in this context means that the individual detector elements do not have to sense contiguous portions of the spectrum, nor do they have to sense evenly spaced portions of the spectrum, nor do they have to be evenly spaced on the substrate, nor must they be arranged in any sort of unit cell configuration repeated dozens or hundreds of times in the sensor. Rather, the detector elements may sense limited wavelength regions of varying widths, and the limited wavelength regions may be irregularly located over the spectrum. The invention when built for a particular application must be designed with the associated optical components in mind. Detector elements are placed on the substrate with regard to how the light to be sensed is incident upon the surface of the substrate (or upon the detector elements sitting upon the substrate) by the associated optical components.

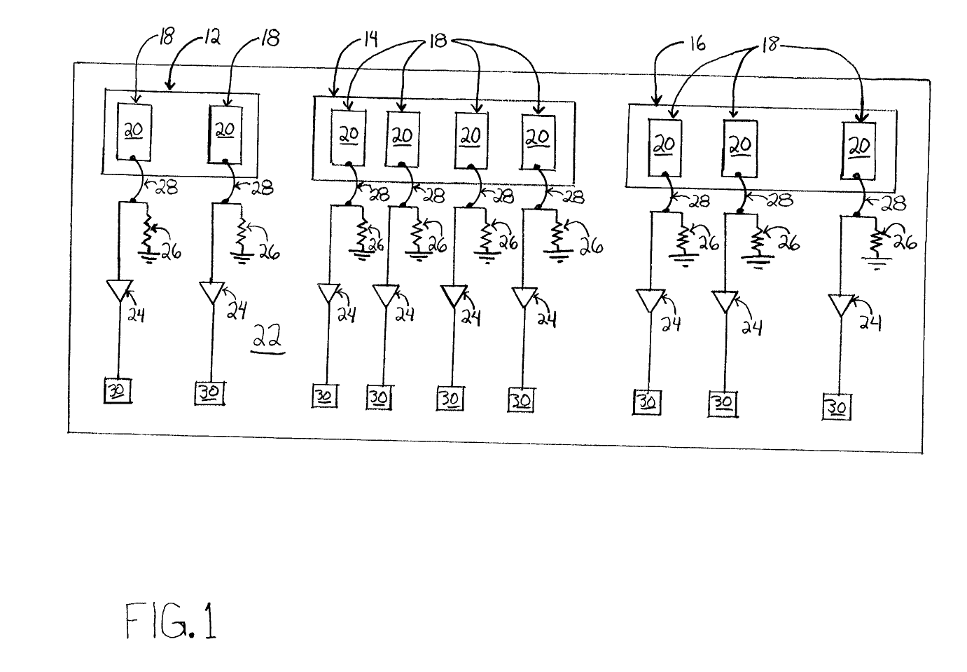

Referring now to FIG. 1, a highly schematic diagram of a composite detector array is shown. The array comprises a first subarray 12, a second subarray 14, and a third subarray 16. Each subarray has one or more detector elements 18. Each detector element 18 is adapted to receive a particular limited wavelength region of light from an external optics system associated with the composite detector array. Each detector element 18 comprises a photosensitive surface 20 which is capable of generating a signal related to the amount of light incident upon the photosensitive surface 20. The first, second, and third subarrays 12, 14, 16 are attached to a substrate 22 at positions corresponding to the wavelength regions of light transmitted by the optics system. FIG. 1 depicts the subarrays 12, 14, 16 arranged in a generally linear fashion, with the detector elements 18 also arranged in a generally linear fashion; however, the detector elements may be arranged over a two dimensional surface to give a planar array if so required by the optics system. The substrate 22 in one embodiment is a silicon semiconductor material having preamplifier circuits 24 and load resistors 26 manufactured into the substrate 22. Each detector element 18 with its photosensitive surface 20 is in electrical communication, e.g. via wire-bond bridges 28, with a preamplifier circuit 24 and a load resistor 26. Each preamplifier circuit 24 is in electrical communication with a contact 30 on the substrate 22 for connection of the detector elements 18 to external components. The signal from the photosensitive surface 20 may thus be transmitted from the detector element 18 to the external components via the preamplifier circuit 24 and the contact 30. The device may have optional addressing circuitry for directing interrogation of the detector elements by the external components. The details of the electronic components on the substrate may vary depending on the number of detector elements on the array. For a large number of detection elements a shift register may be included to allow the detection elements of the array to be interrogated sequentially. Such a design allows signal processing components to be shared, in effect, by multiple detector elements, thereby simplifying manufacture of the arrays. Those of skill in the art of array design are well aware of circuitry for interrogating elements of an array. Other embodiments may have only the first and second subarrays, while still other embodiments may have more than three subarrays. In particular embodiments, each subarray has a composition type that is different from the composition type of any other subarray on the composite detector array.

The detector elements may be any type which is known in the art to be useful for detecting light in the wavelength range of interest, e.g. silicon, germanium, indium-gallium-arsenide, strained indium-gallium-arsenide, indium-arsenide, lead-sulfide, lead-selenide, or mercury-cadmium-telluride. An indium-antimonide detector may also be used in applications where the detector may be cooled to liquid nitrogen temperature.

Arrays of detector elements of a single type and their methods of manufacture are well known. In the current invention, detector elements may be supplied as, e.g. individual photo-sensing diodes, small subarrays of photo-sensing diodes, or any other form of photosensor which may conveniently be included in the detector device. One or more detector elements of a single type may be manufactured on a single subarray, and a plurality of such subarrays are assembled into a single composite detector wherein at least one subarray type is different from at least one other subarray type. Including individual detector elements of the same type onto a single subarray which is then placed on the substrate avoids unneeded complexity in assembly of the detector device. The subarray in this case would have to be designed to have the detector elements spatially arranged to allow the predetermined wavelength range for each detector element to be detected by the appropriate detector element.

At least two, preferably three, or more preferably four detector types are present in the device of the current invention.. There may be at least three, preferably at least four, more preferably at least five, still more preferably at least six, yet more preferably at least eight detector, still again more preferably at least ten, yet again more preferably at least twelve or most preferably at least fifteen individual detector elements placed on the single substrate. A maximum number of detector elements would be about one hundred, preferably no more than forty, still more preferably no more than thirty, yet more preferably no more than twenty-five, still again more preferably no more than twenty, or most preferably no more than eighteen detector elements.

Optical components are used to channel, focus, filter, and/or spectrally disperse the light to be sensed upon the detector elements on the substrate of the detector device. Filters, beam-splitters, slits, windows, lenses, and spectral dispersing elements, may be used individually or in combinations to limit the wavelength range of light reaching a particular detector element. Each detector element is placed upon the substrate in a position that will place each detector element in position to sense a limited wavelength region of light. The size of the limited wavelength regions will vary depending upon several factors. However, in most spectroscopic analysis one expects that the maximum spectral width of the limited wavelength regions will not exceed about 50% of the largest wavelength of light being measured; for example, not more than 1.2 micrometers when the longest wavelength of light being detected is 2.4 micrometers. The minimal size of limited wavelength region would generally be about 1 nm. In one embodiment, the limited wavelength regions are each smaller than about 600 nm wide; in another embodiment, smaller than about 300 nm wide; in still another embodiment, less than about 200 nm wide, and in yet another embodiment, less than about 150 nm wide. The preferred minimal spectral width is somewhat dependent upon the absolute wavelength; at shorter wavelengths, the spectral width may be smaller. For the UV range (˜100 nm to ˜450 nm) each limited wavelength region is generally at least about 5 nm wide. For the visible range (˜450 nm to ˜750 nm) each limited wavelength region is generally at least about 10 nm wide. For the near IR range (˜750 nm to ˜1100 nm) each limited wavelength region is generally at least about 20 nm wide. All detector elements on a substrate may detect limited wavelength regions of equal width, or the widths may differ.

The substrate may be any material that provides sufficient mechanical stability and thermal stability. Thermal stability is important because, as the temperature of the substrate changes, the thermal expansion or contraction of the substrate may shift the relative position of the detector elements, thus shifting the wavelength of light sensed by the detector elements. The substrate material provides a mechanically strong and stable surface for attachment of the detector elements (or subarrays of detector elements) and provides for electrical connection of the detector elements to readout circuitry. Examples of potential substrate materials are aluminum nitride, ceramic materials, polymer materials, or fiberglass materials. The substrate material may form the base of a package, with the package containing contacts for electrical connections and a window or other means of transmitting the light from outside of the package to the detector elements. Alternatively, the substrate may be a material later attached to the base of such a package. Silicon may be useful both as a substrate for attachment of detector elements of other types and as a type of detector element. In such an embodiment, one or more silicon-type detector elements may be fabricated onto a silicon chip with associated circuitry also on the chip; the silicon chip also serves as the substrate for one or more detector elements of differing type to be placed on the chip and electrical connections between the chip and the detector element to be made.

A potential advantage of the configuration of the detector elements of the current invention is that, because only selected portions of the spectrum are read using a limited number of detector elements, the readout circuitry used to read the device may be correspondingly less complex, i.e. using smaller microprocessor, less expensive, low power consumption, the signals need to be sampled at a rapid rate. Where the light to be detected is of low intensity, collecting or focusing light over a broader wavelength range may allow more sensitive detection of the light, because all the light that falls over a, e.g., 50 nm range is collected instead of only the light over a 1 nm range.

Response characteristics are generally improved by cooling the detector elements or maintaining the detector elements at a steady temperature. Cooling some types of photodiodes is know to reduce noise, improve current flow, and improve detectivity. The result is an increase in the diode response. For high-power applications such as pulsed laser detection, cooling is generally not necessary. For sensitive, low-power applications such as temperature measurements, the detector elements should be cooled or at least temperature-stabilized. Stabilizing the temperature near 22° C. room temperature will not improve performance, but will prevent changes in detector response due to ambient temperature drift. Cooling may be conveniently accomplished by including a thermoelectric cooling device, which will allow cooling of the detector elements in the array.

### EXAMPLE

The number of individual detecting elements should be limited in number to ease manufacture. Because the number of individual sensing elements needs to be limited, various statistical methods and model systems were employed to optimize the detector design based on the application of the detector. This analysis has been applied to the sensing of blood glucose concentration in human subjects.

### General Method for Determining the Limited Wavelength Regions Needed for Glucose Analysis

The number and size (wavelength range) of the discrete limited wavelength regions that would be analyzed to accurately determine the glucose composition of a sample is determined by, among other things: 1) the strength of the glucose absorption peak, 2) the strength of the absorption peaks of the interfering species in the sample, 3) the number of interfering species in the sample, 4) the size of the overlap of the absorption peaks of the interfering species with the glucose absorption peak, and 5) the fluctuations in these factors over the range of conditions of the samples. Due to this wide range of conditions that could affect the measurements, an iterative process is used for searching the optimal number and size of the limited wavelength regions. The process is called building a calibration model. The process yields two useful pieces of information: a) a mathematical model for predicting glucose composition of an unknown sample and b) the number and size of the discrete limited wavelength regions for designing the instrument.

The process requires a large number of samples, covering the entire range of conditions that would be encountered in the course of application of the method. The accurate value of glucose concentrations of these samples and their spectra are also required. The glucose values for the samples can be obtained from a standard laboratory glucose-testing device. Similarly, the spectral data could be obtained using a standard laboratory spectrometer. Typically this data set, consisting of the glucose concentrations and the spectra, is divided into a modeling set and an error prediction set. The following steps are used to build the model using these two sets:

1) Identify the major glucose absorption peak in the spectral range to be used. Identify the interfering species that would be present in the samples (“interferents”) and their spectral signatures in the spectral range. It is necessary that the spectral range itself be broad enough to include at least one absorption peak of each of the interfering species. Determine the number and width of the absorption peaks of the interfering species within this spectral range. The spectral width of an absorption feature (e.g. a glucose or interferent absorption peak) needs to be picked wide enough to allow typically at least 99% of the signal associated with this feature to be used in the data analysis (although lower percentages of the signal may be used). This criterion is applied to the chemical species of interest as well as the interfering chemical species. In the first step one assumes that these spectral ranges would suffice to build a calibration model.

2) The next step in building a calibration model could be any of the standard chemometric techniques such as Partial Least Squares (PLS) or Principal Component Resolution (PCR) or Wavelet analysis or any combination of these or other similar techniques. This is an iterative process wherein the spectral data is filtered to use only the data spectral ranges identified in step 1. These data for the samples together with their corresponding glucose values are then used to build the calibration model according to one of the techniques mentioned above. A good reference for these techniques is: Multivariate Calibration, Harald Martens and Tormod Naes, John Wiley & Sons, ISBN 0471909793; U.S. Pat. No. 6,119,026 to McNulty et al. teaches another technique based on wavelet analysis. This process yields a matrix G which can be used to predict glucose concentration matrix Cᵤₙ of unknown samples whose spectral data set is represented by matrix Sᵤₙ using the equation

Cᵤₙ=GSᵤₙ

3) This equation is then used along with the spectral data set for the prediction data set mentioned above. This yields a matrix C_{pred} which contains the predicted glucose concentrations for the samples in the prediction data set. The prediction error or a measure thereof can be determined from this predicted set of concentrations and the actual glucose composition in the prediction data set.

4) One then changes the number of spectral data sets and/or the sizes of the spectral ranges in step 1 systematically until the prediction error calculated in step 3 is minimal. Typically, one would include more absorption peaks for the interfering species in the next iteration. For example, one would add another peak of an interfering species that has considerable overlap with the glucose absorption peak. Such criteria for adding peaks are familiar to the ones in the field of spectroscopy of molecules and groups.

The steps 1-4 represent a typical procedure for determining the optimal number and size of the limited wavelength regions. The teachings of this invention are not restricted to any specific method of optimization.

### Spectral Range of Interest to Glucose Determinations

It is well known that the best optical region for determining glucose composition from spectral analysis of biological samples is 1.0 μm to 2.4 μm. Other spectral regions of interest are 0.8 μm to 1.6 μm and 7.5 μm to 10.5 μm. Interferents in the system may strongly absorb light in other portions of the spectrum, which emphasizes the importance of having a detector which is a composite of several detector types to detect wavelengths over a wide range of the light spectrum.

In this example, the spectral ranges chosen for having potential utility for glucose measurement are 1) 600-900 nm, 2) 1400-1800 nm and 3) 2100-2400 nm. Light in the spectral range 1) 600-900 nm is detected using Silicon photo-detectors. The spectral range 2) 1400-1800 nm is covered by using photo-detectors of a moderately strained InGaAs type. Calculations indicate that about 0.8% compressive strained InGaAs surrounded by suitable InGaAsP barriers (in an Multi-Quantum Well absorption structure) will give excellent detectors with low leakage currents. Finally, to cover the spectral range 3) 2100-2400 nm the amount of strain needed is quite large and detectors with partially relaxed regions with high indium composition may be used. Detectors of this type may result in higher leakage currents. An alternative in this spectral region is to use InAs detectors. The three different material systems considered for this example would of course need to be tweaked to achieve the appropriate level of performance. The number of detector elements, their positions on the substrate, and the particular limited wavelength regions of light to be measured by the detector elements may be established (and tweaked for optimum performance) from calibration studies, as described above, for example.

For purposes of assembling the complete detector system with improved noise behavior, a common silicon carrier on which the necessary electronics (pre-amplifiers, resistors, etc) are integrated using standard silicon integrated circuit technology is employed. Onto this common carrier the three (for the glucose example considered in this example) detector element subarrays are placed and die-attached. The appropriate device pads are wire-bonded to complete the detector assembly. The detector assembly may then be incorporated into an instrument having the necessary light source(s), optical components, and data processing capabilities to perform a specified test measurement.

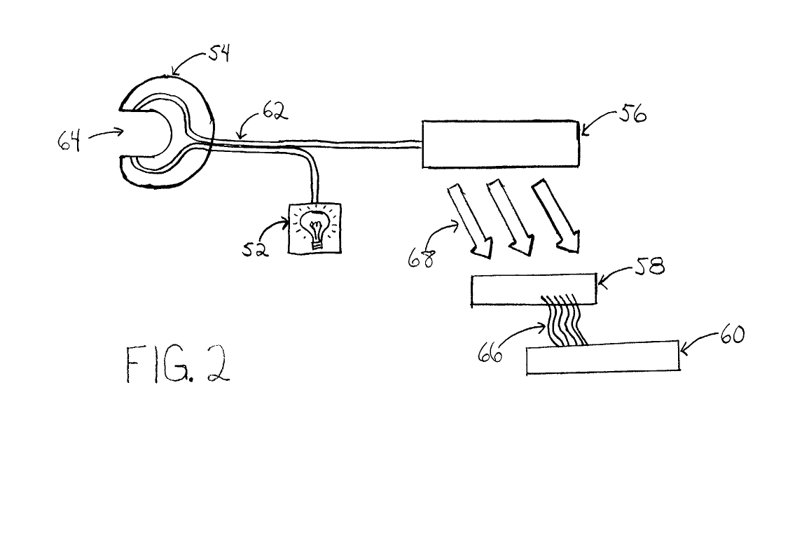

As shown in FIG. 2, in one embodiment, the non-invasive measurement of the concentration of glucose in blood is performed with at least one light source 52, a fiber-optic probe 54, a spectral dispersing element 56, a composite detector device 58, and a microprocessor 60 for receiving and processing information from the composite detector device 58. The fiber optic probe 54 consists of a dual light conductor 62 which is used in either the transmission or scattering mode. Light from the light source 52 is transmitted through one of the dual conductors, which terminates at a sampling site 64 on the optical probe 54. The sampling site 64 is adapted for positioning a portion of the patient's body for transmission of light into the body. The light transmitted into the body undergoes scattering and characteristic absorption depending on the identity of the chemical species present. A portion of the light having undergone scattering and absorption is back scattered from the body and collected and transmitted back to the spectral dispersing element 56 by the other fiber-optic conductor. The spectral dispersing element 56 transmits light 68 to the composite detector device 58. The fiber optic probe 54, placed in contact with the body, is arranged so that either a transmission or a scattering measurement is performed. In the transmission mode, the fiber-optic probe 54 is arranged so that the light from the source 52 can be passed through the portion of the body, which may be, e.g., the ear lobe, tongue, cheek, or webbing between the fingers or toes, and its spectral absorption characteristics measured. This is accomplished by placing the body section between the opposing ends of the dual fiber so that light from the fiber-optic conductor connected to the light source 52 passes through the body section to the other fiber-optic conductor which transmits the attenuated light to the spectral dispersing element 56 and the composite detector device 58. In the scattering mode, a bifurcated fiber-optic probe is used. The bifurcated probe consists of two separate bundles of fibers, one bundle being centrally located and the other bundles being disposed in any configuration surrounding the central bundle. To measure blood glucose, the sensing end of the probe is placed in direct contact with an outer surface of the body. Light from the fibers connected to the light source is transmitted through that portion of the body undergoing both characteristic spectral absorption and scattering. Some of the scattered light which has traveled through the body experiencing absorption is collected by the optical fibers in the configuration and then transmitted to the spectral dispersing element 56 and composite detector 58.

The light source 52 in this embodiment may be any one or more wide spectrum sources, e.g. incandescent lamps, or may be multiple narrow band sources, e.g. light emitting diodes. Such sources are known in the art. The spectral dispersing element 56 for this embodiment can include any apparatus which allows specified wavelength regions of light to be localized at particular sites. Examples are well known in the art, such as a prism, diffraction grating, or a set of optical filters. Other optical components may be included, such as waveguides, lenses, slits, or lightsplitting elements. The purpose of the spectral dispersing element 56 is to disperse the light passing through the body into its spectral components to distinguish and quantify those particular spectral components that may be used to measure blood glucose. The characteristic light absorption by the glucose can be related directly to its concentration in blood. The apparatus may, in some embodiments, be altered to allow the apparatus to detect fluorescence or optical rotation.

The microprocessor 60 receives the output signal from the composite detector 58 via the electrical connectors 66, calculates the concentration of blood glucose, and formats the output to a display or recording device giving blood glucose concentration in selected units. Besides being used to perform data processing functions, the microprocessor may be used to control the operation of the instrument.

Composite detector devices as described in this disclosure may be used for a variety of applications besides blood glucose monitoring. The design of the optics system and the detector device may be geared towards other non-invasive or invasive blood analyte testing, e.g. blood gases, pH, potassium, lipid, ketone, cholesterol, bile salt, to name a few. Composite detector devices may also be used for non-clinical applications, such as environmental testing or wastewater testing.

Although the above-described embodiments of the present invention have been described in detail, various modifications to the present invention will become apparent to those skilled in the art from the foregoing description and accompanying drawings and will be within the scope of the invention, which is to be limited only by the following claims.

What is claimed is:

1. A device for detecting light from a sample, wherein said light is spatially dispersed in a wavelength dependent manner, the device comprising a plurality of detector elements upon a single substrate, each detector element being located on the substrate such that each said detector element receives a limited wavelength region of the light, the number of detector elements and the number of limited wavelength regions being between 2 and 100.

2. The device of claim 1, wherein at least one detector element has a composition selected from the group consisting of silicon, germanium, indium-gallium-arsenide, strained indium-gallium-arsenide, indium-arsenide, lead-sulfide, lead-selenide, mercury-cadmiumtelluride, and indium-antimonide.

3. The device of claim 1, wherein the composition of one detector element is different from the composition of at least one other detector element.

4. The device of claim 1, wherein the substrate is upon a thermoelectric cooling material.

5. The device of claim 1, wherein each limited wavelength region is between 1 nanometer and 1.2 micrometers wide.

6. The device of claim 1, further comprising a packaging material substantially surrounding the detector elements upon the substrate, said packaging material having at least one opening allowing transmission of light to the detector elements.

7. The device of claim 1, wherein the number of detector elements and number of limited wavelength regions are selected to optimize information obtained about the sample while limiting or minimizing the number of detector elements present on the substrate.

8. The device of claim 1, wherein each detector element may be separately interrogated.

9. An instrument comprising the device of claim 1, wherein the instrument noninvasively measures a blood chemistry value of a subject.

10. The instrument of claim 9, wherein the blood chemistry value measured is selected from the group consisting of blood glucose concentration, blood gases, pH, potassium concentration, lipid concentration, ketone concentration, cholesterol concentration, and bile salt concentration.

11. A method of obtaining a blood chemistry measurement from a subject, the method comprising the steps of
a) shining light from at least one light source upon the body of a subject,
b) receiving light from the body of the subject which contains information about the blood chemistry of the subject,
c) focusing and/or filtering the light received from the body of the subject,
d) casting the focused and/or filtered light upon a composite detector device, said composite detector device having a limited number of detector elements, each detector element receiving a limited wavelength region of the light, wherein the limited wavelength region of light received by each detector element is predetermined for optimizing the information obtained about the blood chemistry of the subject while reducing or minimizing the number of detector elements in the composite detector device,
e) receiving information about the blood chemistry of the subject from the composite detector device, said information being about a limited number of wavelength regions of light,
f) using the information received in step e) to calculate the blood chemistry measurement from the subject.

12. The method of claim 11, wherein multiple narrow-band light sources are used.

13. The method of claim 11, wherein step c) includes spatially separating the light in a wavelength dependent manner.

14. The method of claim 11, wherein each limited wavelength region of light is between 1 nanometer and 1.2 micrometers wide.

15. The method of claim 11, wherein the blood chemistry measurement is blood glucose concentration, the number of optical sensing elements is at least three, and the limited wavelength region of light received by the at least three optical sensing elements are located between about 600 nm and about 900 nm, between about 1400 and about 1800 nm, and between about 2100 and about 2400 nm, respectively.

16. A method of designing a composite detector device for obtaining information about a sample, the method comprising the steps of
a. using a detector to obtain spectral information about the sample at varying conditions of concentration, temperature, and varying amounts of additional materials in the sample which also produce a spectral signal at wavelengths used to study the sample,
b. using multivariate analysis of the spectral information obtained in step a) to optimize the number of detector elements, the wavelength region sensed by each detector element, and the position of the detector element in the composite detector device.
